(19) United States
(12) Patent Application Publication
(10) Pub. No.: US 2010/0113616 A1
(43) Pub. Date: May 6, 2010

(54) INHIBITION OF DISCOLORATION BY WASHING AND CLEANING AGENTS AND/OR COSMETIC AGENTS

(76) Inventors: Thomas Gerke, Neuss (DE); Gerard Veldman, (US); Andreas Bauer, Kaarst (DE); Werner Faber, Willich (DE)

Correspondence Address: Henkel Corporation, 10 Finderne Avenue, Bridgewater, NJ, 08807, US

(21) Appl. No.: 12/613,767

(22) Filed: Nov. 6, 2009

Related U.S. Application Data
(63) Continuation of application No. PCT/EP08/55242, filed on Apr. 29, 2008.

(30) Foreign Application Priority Data
May 8, 2007 (DE) DE102007022 069.5

(51) Int. Cl. C11D 3/50; A61K 8/20; C11D 3/16; A61K 8/36; A61Q 5/02; A61Q 19/10

(52) U.S. Cl. 514/769; 514/772; 510/102; 510/101; 510/106; 510/119; 510/130; 510/491; 514/784

(57) ABSTRACT

Method of inhibiting discoloration in agents by incorporating iodide salt(s), preferably calcium, potassium and/or sodium iodides into the formulation of those agents. The method is particularly suited for agents comprising vanillin and/or vanillin derivatives, wherein vanillin and/or vanillin derivatives are components of a fragrance mixture and the agents are washing and cleaning agents or cosmetics agents.

The present application is a continuation of PCT/EP2008/055242, filed 29 Apr. 2008, which claims the benefit of DE 102 007 022 069.5, filed 8 May 2007, each of which is incorporated herein by reference in their entirety.

The present invention relates to the use of iodide salt(s), preferably calcium, potassium and/or sodium iodides, as discoloration inhibitor (inhibitors) for vanillin- and/or vanillin-derivative-containing agents, wherein vanillin and/or vanillin derivatives are components of a fragrance mixture and the agents are selected from the group of washing and cleaning agents or cosmetic agents.

Vanillin is the main aroma substance contained in vanilla and is a natural aroma substance. Vanillin is commonly found in nature and is known, inter alia, as a component of essential oils and natural aromas. Vanillin is most commonly found in Tahitian vanilla capsules (Vanilla planifolia), but is also found in storax, cloves and other plants. It generally decomposes slowly under the influence of light and humidity and becomes brown-colored (formation of dehydrodivanillin or an oxidation reaction leading to vanillic acid). The use of vanillin in agents, in particular washing or cleaning agents and especially in soap perfumes is therefore problematic (discoloration of the final product). The addition of vanillin or vanillin derivatives to soaps or washing and cleaning agents is therefore generally avoided in order to prevent discoloration of the product. As an alternative to this approach, attempts have hitherto also been made to inhibit the discoloration of vanillin-containing products by adding different antioxidants. However, neither option has been successful in solving the problem caused by vanillin or vanillin derivatives in agents such as soaps.

It was accordingly the object of the present invention to identify and provide compounds or substances which suppress or inhibit the discoloration of vanillin- and/or vanillin-derivative-containing products to the greatest possible extent. A further object of the invention was to incorporate these compounds or substances into washing and cleaning agents or cosmetic agents such as soaps without changing their other properties, such as the impression of the fragrance (olfactory stability) or the stability of the formulation.

Several million bars of soap are sold every year in Germany alone for personal hygiene purposes. The market requirements for these mass-produced consumer goods are becoming ever more demanding—bars of soap must not only clean but also nourish the skin, that is to say prevent it from becoming dry, replenish the lipids in the skin and provide protection against external influences. The soap is also expected to be particularly compatible with skin, but must nevertheless produce copious amounts of creamy suds and have a pleasant feel on the skin. Soap manufacturers are therefore constantly searching for new ingredients which meet these increasingly stringent requirements.

It has surprisingly now been found that the use of iodide salt(s) retards or inhibits the discoloration of products into which vanillin and vanillin derivatives have been incorporated. In particular, it has been shown that the use of iodide salts enables soap discoloration to be successfully prevented. Moreover, it has also been shown that the olfactory properties of the soaps remain stable and do not change over a prolonged period of time.

The present invention therefore relates to the use of iodide salt(s) as a discoloration inhibitor for vanillin- and/or vanillin-derivative-containing agents. Vanillin and/or vanillin derivatives in this case are preferably components of a fragrance mixture.

Vanillin or vanillin derivatives are to be understood in the context of the present invention as compounds according to formula I:

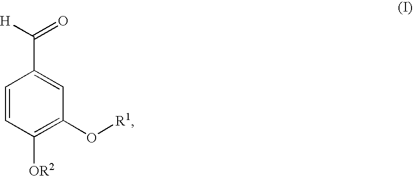

wherein R¹ is a methyl, ethyl or propyl residue and R² is hydrogen, a C₁-C₃ alkyl residue or —C(O)—R³, wherein R³ is an alkyl residue having 1 to 5 C atoms, preferably methyl, ethyl, n-propyl, isopropyl or butyl.

In a preferred embodiment, R² is hydrogen or —C(O)—R³, wherein R³ is an isopropyl residue in this case.

Preferred compounds according to formula I are selected from 4-hydroxy-3-methoxy-benzaldehyde (R¹=methyl, R²=H), 4-hydroxy-3-ethoxy-benzaldehyde (R¹=ethyl, R²=H) and hydroxy-3-methoxy-benzaldehyde-2-methylpropionate (R¹=methyl, R²=—C(O)—CH(CH₃)₂).

The iodide salts used according to the invention are preferably employed in fragrance mixtures. A fragrance mixture preferably encompasses different perfumes which may be selected from the group of essential oils, perfume aldehydes, perfume ketones and/or perfume esters. According to the invention, a fragrance mixture of this type contains at least one compound according to formula I. The fragrance mixture preferably contains at least one compound selected from 4-hydroxy-3-methoxy-benzaldehyde, 4-hydroxy-3-ethoxy-benzaldehyde and hydroxy-3-methoxy-benzaldehyde-2-methylpropionate. The fragrance mixture may obviously also contain a mixture of a plurality of the aforementioned vanillin derivatives according to formula I.

Fragrances and perfumes are to be understood as synonyms in the meaning of the present invention. Any conventional fragrance aldehydes, fragrance ketones and fragrance esters which typically contribute to a pleasant odor may be used as further fragrance aldehydes, fragrance ketones or fragrance esters which may be contained in the fragrance mixture.

According to the invention, “fragrance ketones” are fragrances having at least one free keto group. Mixtures of different ketones may also be used. Preference is given to fragrance ketones selected from the group encompassing Buccoxime, isojasmone, methyl beta-naphthyl ketone, musk indanone, tonalide/musk plus, alpha-damascone, beta-damascone, delta-damascone, iso-damascone, damascenone, damask rose, methyl dihydrojasmonate, menthone, carvone, camphor, fenchone, alpha-ionone, beta-ionone, dihydro-beta-ionone, fleuramone, dihydrojasmone, cis-jasmone, Iso E Super (1-(1,2,3,4,5,6,7,8-octahydro-2,3,8,8-tetramethyl-2-naphthalenyl)-ethan-1-one and the respective isomers), methyl cedrenyl ketone or methyl cedrylone, acetophenone, methylacetophenone, para-methoxy acetophenone, methyl beta-naphthyl ketone, benzylacetone, benzophenone, para-hydroxy phenyl butanone, celery ketone or livescone, 6-isopropyldecahydro-2-naphthone, dimethyloctenone, Freskomenth, 4-(1-ethoxyvinyl)-3,3,5,5,-tetramethylcyclohexanone, methylheptanone, 2-(2-(4-methyl-3-cyclohexen-1-yl)propyl)-cyclopentanone, 1-(p-menthen-6(2)-yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, Dulcinyl or Cassione, gelsone, hexylone, isocyclemone E, methyl cyclocitrone, methyl lavender ketone, orivone, para-tert-butylcyclohexanone, verdone, delphone, muscone, neobutenone, plicatone, veloutone, 2,4,4,7-tetramethyl-oct-6-en-3-one, tetrameran, hedione and mixtures thereof. The ketones may preferably be selected from alpha-damascone, delta-damascone, iso-damascone, carvone, gamma-methyl ionone, Iso E Super, 2,4,4,7-tetramethyl-oct-6-en-3-one, benzylacetone, beta-damascone, damascenone, methyl dihydrojasmonate, methyl cedrylone, hedione and mixtures thereof.

According to the invention, “fragrance aldehydes” are fragrances having at least one free aldehyde group. Suitable fragrance aldehydes may be any aldehydes which, in a similar manner to fragrance ketones, provide a desired fragrance or a fresh sensation. The fragrance aldehyde may be a single aldehyde or a mixture of aldehydes. The following are cited as preferred examples from the large group of fragrance aldehydes: octanal, citral, melonal, Lilial, floralozone, canthoxal, 3-(4-ethylphenyl)-2,2-dimethylpropanal, 3-(4-methoxyphenyl)-2-methylpropanal, 4-(4-hydroxy-4-methylpentyl)-3-cyclohexene-1-carboxaldehyde, phenylacetaldehyde, methyl nonyl acetaldehyde, 2-phenylpropan-1-al, 3-phenylprop-2-en-1-al, 3-phenyl-2-pentylprop-2-en-1-al, 3-phenyl-2-hexylprop-2-enal, 3-(4-isopropylphenyl)-2-methylpropan-1-al, 3-(4-ethylphenyl)-2,2-dimethylpropan-1-al, 3-(4-tert-butylphenyl)-2-methyl-propanal, 3-(3,4-methylenedioxyphenyl)-2-methylpropan-1-al, 3-(4-ethylphenyl)-2,2-dimethylpropanal, 3-(3-isopropylphenyl)butan-1-al, 2,6-dimethylhept-5-en-1-al, n-decanal, n-undecanal, n-dodecanal, 3,7-dimethyl-2,6-octadien-1-al, 4-methoxybenzaldehydes, 3-methoxy-4-hydroxybenzaldehydes, 3-ethoxy-4-hydroxybenzaldehydes, 3,4-methylenedioxybenzaldehyde and 3,4-dimethoxybenzaldehyde, adoxal, anisaldehyde, cumal, ethylvanillin, Florhydral, helional, heliotropin, hydroxycitronellal, Koavon, lauryl aldehyde, Lyral, methyl nonyl acetaldehyde, bucinal, phenylacetaldehyde, undecyl aldehyde, vanillin, 2,6,10-trimethyl-9-undecenal, 3-dodecen-1-al, alpha-n-amylcinnamaldehyde, 4-methoxybenzaldehyde, benzaldehyde, 3-(4-tert-butylphenyl)-propanal, 2-methyl-3-paramethoxyphenyl propanal, 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzaldehyde, 1,2,3,4,5,6,7,8-octahydro-8,8-dimethyl-2-naphthaldehyde, 2,4-dimethyl-3-cyclohexene-1-carboxaldehyde, 1-decanal, decylaldehyde, 2,6-dimethyl-5-heptenal, 4-(tricyclo[5.2.1.0(2,6)]-decylidene-8)-butanal, octahydro-4,7-methano-1-indene carboxaldehyde, 3-ethoxy-4-hydroxybenzaldehyde, para-ethyl-alpha,alpha-dimethyl hydrocinnamaldehyde, alpha-methyl-3,4-(methylenedioxy)-hydrocinnamaldehyde, 3,4-methylenedioxybenzaldehyde, alpha-n-hexylcinnamaldehyde, m-cumene-7-carboxaldehyde, alpha-methylphenylacetaldehyde, 7-hydroxy-3,7-dimethyloctanal, undecenal, 2,4,6-trimethyl-3-cyclohexene-1-carboxaldehyde, 4-(3)(4-methyl-3-pentenyl)-3-cyclohexene carboxaldehyde, 1-dodecanal, 2,4-dimethyl-cyclohexene-3-carboxaldehyde, 4-(4-hydroxy-4-methylpentyl)-3-cylohexene-1-carboxaldehyde, 7-methoxy-3,7-dimethyloctan-1-al, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexene-1-carboxaldehyde, 5- or 6-methoxyhexahydro-4,7-methanoindane-1 or 2-carboxaldehyde, 3,7-dimethyloctan-1-al, 1-undecanal, 10-undecen-1-al, 4-hydroxy-3-methoxybenzaldehyde, 1-methyl-3(4-methylpentyl)-3-cyclohexene carboxaldehyde, 7-hydroxy-3,7-dimethyl-octanal, trans-4-decenal, 2,6-nonadienal, para-tolyl acetaldehyde, 4-methylphenyl acetaldehyde, 2-methyl-4-(2,6,6-trimethyl-1-cyclohexen-1-yl)-2-butenal, ortho-methoxycinnamaldehyde, 3,5,6-trimethyl-3-cyclohexene carboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanoindane-1-carboxaldehyde, 2-methyloctanal, alpha-methyl-4-(1-methylethyl)benzeneacetaldehyde, 6,6-dimethyl-2-norpinene-2-propionaldehyde, para-methylphenoxy acetaldehyde, 2-methyl-3-phenyl-2-propen-1-al, 3,5,5-trimethylhexanal, hexahydro-8,8-dimethyl-2-naphthaldehyde, 3-propyl-bicyclo[2.2.1]-hept-5-ene-2-carbaldehyde, 9-decenal, 3-methyl-5-phenyl-1-pentanal and methyl nonyl acetaldehyde.

For further suitable fragrances selected from the groups of aldehydes and ketones, reference is made to Steffen Arctander, published 1960 and 1969 respectively, reprinted 2000, ISBN: Aroma Chemicals Vol. 1: 0-931710-37-5, Aroma Chemicals Vol. 2: 0-931710-38-3.

Furthermore, individual perfume compounds of natural or synthetic origin, for example of the ester, ether, alcohol and hydrocarbon types, may be used as perfume oils or fragrances. Perfume compounds of the ester type include, for example, benzyl acetate, phenoxyethyl isobutyrate, p-tert-butylcyclohexyl acetate, linallyl acetate, dimethyl benzyl carbinyl acetate (DMBCA), phenyl ethyl acetate, benzyl acetate, ethyl methylphenylglycinate, allyl cyclohexyl propionate, styrallyl propionate, benzyl salicylate, cyclohexyl salicylate, floramate, melusate and jasmacyclate. Examples of ethers include benzyl ethyl ether and ambroxan, examples of alcohols include anethole, citronellol, eugenol, geraniol, linalool, phenyl ethyl alcohol and terpineol. The hydrocarbons are primarily terpenes such as limonene and pinene. However, mixtures of different perfumes which produce a pleasant fragrance when combined are preferred.

Perfume oils of this type may also contain natural perfume mixtures which can be obtained from plant sources such as pine, citrus, jasmine, patchouli, rose or ylang-ylang oil. Clary sage oil, chamomile oil, clove oil, melissa oil, mint oil, cinnamon leaf oil, lime blossom oil, juniper berry oil, vetiver oil, olibanum oil, galbanum oil and labdanum oil as well as orange blossom oil, neroli oil, orange peel oil and sandalwood oil are also suitable. Further conventional perfumes which may be used within the scope of the present invention include, for example, essential oils such as angelica root oil, anise oil, arnica blossom oil, basil oil, bay oil, champaca blossom oil, abies alba oil, abies alba cone oil, elemi oil, eucalyptus oil, fennel oil, pine needle oil, galbanum oil, geranium oil, ginger grass oil, guaiac wood oil, gurjun balsam oil, helichrysum oil, ho oil, ginger oil, Iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, melissa oil, musk seed oil, myrrh oil, clove oil, neroli oil, niaouli oil, olibanum oil, oregano oil, palmarosa oil, patchouli oil, balsam Peru oil, petitgrain oil, black pepper oil, peppermint oil, allspice oil, pine oil, rose oil, rosemary oil, sandalwood oil, celery oil, spike oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, citrus oil and cypress oil as well as ambrettolide, ambroxan, α-amylcinnamaldehyde, anethole, anisaldehyde, anise alcohol, anisole, anthranilic acid methyl ester, acetophenone, benzyl acetone, benzaldehyde, ethyl benzoate, benzophenone, benzyl alcohol, benzyl acetate, benzyl benzoate, benzyl formate, benzyl valerate, borneol, bornyl acetate, boisambrene forte, α-bromostyrene, n-decyl aldehyde, n-dodecylaldehyde, eugenol, eugenol methyl ether, eucalyptol, farnesol, fenchone, fenchyl acetate, geranyl acetate, geranyl formate, heliotropin, heptin carboxylic acid methyl ester, heptanal, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl-n-amylketone, methylanthranilic acid methyl ester, p-methylacetophenone, methyl chavicol, p-methylquinoline, methyl-β-naphthylketone, methyl-n-nonyl acetaldehyde, methyl-n-nonyl ketone, muscone, β-naphthol ethyl ether, β-naphthol methyl ether, nerol, n-nonyl aldehyde, nonyl alcohol, n-octyl aldehyde, p-oxy-acetophenone, pentadecanolide, β-phenyl ethyl alcohol, phenylacetaldehyde-dimethyl acetal, phenyl acetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, γ-undelactone, vanillin, veratric aldehyde, cinnamaldehyde, cinnamyl alcohol, cinnamic acid, ethyl cinnamate, benzyl cinammate, diphenyl oxide, limonene, linalool, linallyl acetate and propionate, melusate, menthol, menthone, methyl-n-heptenone, pinene, phenylacetaldehyde, terpinyl acetate, citral, citronellal and mixtures thereof.

A fragrance mixture preferably encompasses perfumes selected from the group of jasmones, ionones, damascones and damascenones, menthone, carvone, Iso E Super (1-(1,2,3,4,5,6,7,8-octahydro-2,3,8,8-tetramethyl-2-naphthalenyl)-ethan-1-one and the respective isomers), methylheptenones, melonal, cymene, Helional, hydroxycitronellal, Koavone, methyl nonyl acetaldehyde, phenylacetaldehyde, undecyl aldehyde, 3-dodecen-1-al, alpha-n-amylcinnamaldehyde, benzaldehyde, 3-(4-tert-butylphenyl)-propanal, 2-methyl-3-(paramethoxyphenyl propanal), 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzaldehyde, 2,4-dimethyl-3-cyclohexene-1-carboxaldehyde, 2-methyl-3-(isopropylphenyl) propanal, decylaldehyde, 2,6-dimethyl-5-heptenal, alpha-n-hexylcinnamaldehyde, 7-hydroxy-3,7-dimethyloctanal, undecenal, 2,4,6-trimethyl-3-cyclohexene-1-carboxaldehyde, 1-dodecanal, 2,4-dimethyl-cyclohexene-3-carboxaldehyde, 4-(4-hydroxy-4-methylpentyl)-3-cylohexene-1-carboxaldehyde, 2-methylundecanal, 2-methyldecanal, 1-nonanal, 1-octanal, 2,6,10-timethyl-5,9-undecadienal, 2-methyl-3-(4-tert-butyl)propanal, dihydrocinnamaldehyde, 3,7-dimethyloctan-1-al, 1-undecanal, 10-undecen-1-al, 4-hydroxy-3-methoxybenzaldehyde, trans-4-decenal, 2,6-nonadienal, para-tolyl acetaldehyde, 3,7-dimethyl-2-methylene-6-octenal, 2-methyloctanal, alpha-methyl-4-(1-methylethyl)benzeneacetaldehyde, 2-methyl-3-phenyl-2-propen-1-al, 3,5,5-trimethylhexanal, 3-propyl-bicyclo[2.2.1]-hept-5-ene-2-carbaldehyde, 9-decenal, 3-methyl-5-phenyl-1-pentanal, methyl nonyl acetaldehyde, citral, 1-decanal, Florhydral, 2,4-dimethyl-3-cyclohexene-1-carboxaldehyde and heliotropin.

In a preferred embodiment, the iodide salts preferably used according to the invention are employed in a fragrance mixture which is preferably incorporated into washing and cleaning agents or cosmetic agents.

In this embodiment, the washing and cleaning agents are preferably liquid or gel cleaners, softeners, washing agents, all-purpose cleaners, as well as cosmetic agents for hair or skincare such as creams, lotions, oils, gels, soaps and shampoos.

The washing and cleaning agents and cosmetic agents may obviously contain further conventional ingredients of washing and cleaning agents and cosmetic agents. The conventional washing and cleaning agent ingredients are preferably selected from the group of surfactants, builders, bleaching agents, enzymes and other active substances.

The iodide salts used according to the invention are preferably incorporated into solid (washing, cleaning and cosmetic) agents, preferably soaps, where the problem of product discoloration is at its greatest.

The invention therefore further relates to washing and cleaning agents and/or cosmetic agents containing iodide salts in combination with vanillin and/or vanillin derivatives. As mentioned above, the washing and cleaning agents are liquid or gel cleaners, softeners, washing agents and all-purpose cleaners and the cosmetic agents are skin creams, skin lotions, skin oils, gels, soaps and shampoos. Soaps containing iodide salts in combination with vanillin and/or vanillin derivatives are more particularly preferred.

The iodide salts used according to the invention in the agents are preferably alkali metal iodides. The alkali metal iodides are preferably selected from calcium, potassium and sodium iodide and are preferably used in a total amount of from 0.05 to 5% by weight, preferably 0.1 to 2% by weight, in the total composition.

Toilet soap is one of the main types of soap used for personal hygiene. There are two different types of toilet soaps—solid soaps, generally in bar form, and liquid soaps. Accordingly, in a preferred embodiment, the soaps, in which the iodide salts used according to the invention are incorporated, are in the form of shaped bodies and contain other ingredients in addition to surface-active ingredients (surfactants).

In a preferred embodiment, the most important ingredients of shaped bodies of this type are the alkali salts of the fatty acids of natural oils and fats, preferably having chains of 12 to 18 Carbon atoms. Fatty acids of this type are preferably obtainable from coconut oil, palm kernel oil or babassu oil by saponification or cleavage and separation of the shorter-chained components.

Mixtures of the aforementioned fatty acids and fatty acids obtained from beef fat, palm oil and other animal or vegetable fats and oils such as soybean oil, sunflower oil, rape oil, linseed oil and peanut oil, are also suitable. Since lauric acid soaps exhibit particularly good sudsing properties, coconut and palm kernel oils, which are rich in lauric acid, are the preferred raw materials for toilet soap production.

Na-salts of the fatty acid mixtures are solid (curd soaps, soda soaps, toilet soaps) whereas the K-salts are soft and pasty (soft soaps, potassium soaps). In order to allow saponification, the diluted sodium or potassium hydroxide solution is added to the fatty raw materials in such a stoichiometric ratio that there is an excess of lye of at most 0.05% in the finished soap. Nowadays, many soaps are no longer produced directly from fats but from fatty acids obtained by lipolysis. Besides surfactants, other conventional soap additives include fatty acids, fatty alcohols, lanolin, lecithin, vegetable oils such as almond oil, partial glycerides including fat-like substances for replenishing lipids (superfatting agents) in the cleaned skin, antioxidants such as ascorbil palmitate or tocopherol for preventing autoxidation of the soap (rancidity), complexing agents such as nitriloacetate for binding traces of heavy metals which could catalyze the autoxidative decay reaction, perfume oils to produce the desired fragrance, dyes for dyeing the soap, suds-enhancing additives, cosmetic active ingredients for the skin, antimicrobial active ingredients and optionally other specific additives.

Liquid soaps are based both on the K-salts of natural fatty acids and on synthetic anionic surfactants. They contain, in an aqueous solution, fewer surface-active ingredients than solid soaps but contain conventional additives, as well as optional viscosity-regulating components and pearlescing additives. They are preferentially provided in dispensers in public washrooms and the like since they are easy and hygienic to use. Washing lotions for particularly sensitive skin are based on synthetic surfactants with mild activity and additions of substances which nourish the skin and are pH neutral or slightly acidic (pH 5.5).

Alkyl ether sulfates and/or fatty acid alkanolamides are preferably used as suds regulators. Alkyl ether sulfates have a lime-soap-dispersing effect and thus improve sudsing characteristics and suds stability in hard water in particular.

Fatty acid alkanolamides are strong suds boosters and increase the stability of the suds against exposure to fat and soil.

Suitable preferred alkyl ether sulfates employed in the soaps used according to the invention include, for example, alkali or alkanol ammonium salts of sulfuric acid semi-esters of the addition products of 1 to 10 mol ethylene oxide to linear or predominantly linear alcohols having 10 to 18 carbon atoms. Alkyl ether sulfates, the sodium salts of linear, primary C₁₂-C₁₆ fatty alcohol polyglycol ether sulfates having 2 to 4 glycol ether groups are particularly suitable.

Suitable preferred fatty acid alkanolamides are the monoethanolamides and diethanolamides of C₁₂-C₁₈ fatty acids, such as those of coco-fatty acid fractions, palm kernel fatty acid fractions, tallow fatty acids, hydrogenated tallow fatty acids, vegetable fatty acids such as palm oil fatty acid, soya oil fatty acid, sunflower oil fatty acid or mixtures of the aforementioned fatty acids. Coco-fatty acid monoethanolamide and coco-fatty acid diethanolamide are particularly preferred.

Surfactants are generally the main component of the major ingredients of soaps and washing and cleaning agents.

Depending on the purpose thereof, these surface-active substances are obtained from the group of anionic, non-ionic, zwitterionic or cationic surfactants, wherein anionic surfactants are far preferable for cost reasons and on account of their performance in washing and cleaning processes.

In principle, any anionic surface-active agents suitable for use on the human body are suitable as anionic surfactants. These are characterized by a water-solubilizing, anionic group such as a carboxylate, sulfate, sulfonate or phosphate group and a lipophilic alkyl group having approximately 8 to 30 C atoms. Furthermore, glycol or polyglycol ether groups, ester, ether and amide groups as well as hydroxyl groups may be contained in the molecule. Examples of suitable anionic surfactants include the following, each in the form of the sodium, potassium, ammonium as well as mono-, di- and trialkanolammonium salts thereof having 2 to 4 C atoms in the alkanol group,
linear and branched fatty acids having 8 to 30 C atoms (soaps),
ether carboxylic acids of the formula R¹⁴—O—(CH₂—CH₂O)ₓ—CH₂—COOH in which R¹⁴ is a linear alkyl group containing 8 to 30 C atoms and x=0 or 1 to 16,
acyl sarcosides having 8 to 24 C atoms in the acyl group, acyl taurides having 8 to 24 C atoms in the acyl group,
acyl isethionates having 8 to 24 C atoms in the acyl group, sulfosuccinic acid mono- and dialkyl esters having 8 to 24 C atoms in the alkyl group and sulfosuccinic monoalkyl polyoxyethyl esters having 8 to 24 C atoms in the alkyl group and 1 to 6 oxyethyl groups,
linear alkanesulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R¹⁵—O(CH₂—CH₂O)ₓ—OSO₃H, in which R¹⁵ is a preferably linear alkyl group having 8 to 30 C atoms and x=0 or 1 to 12,
mixtures of surface-active hydroxy sulfonates,
sulfated hydroxyalkyl polyethylene and/or hydroxyalkylene propylene glycol ethers,
sulfonates of unsaturated fatty acids having 8 to 24 C atoms and 1 to 6 double bonds,
esters of tartaric acid and citric acid containing alcohols which are addition products of approximately 2 to 15 molecules of ethylene oxide and/or propylene oxide to fatty alcohols containing 8 to 22 C atoms,
alkyl and/or alkenyl ether phosphates of formula (E1-I)—

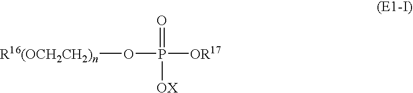

in which R¹⁶ preferably represents an aliphatic hydrocarbon residue having 8 to 30 carbon atoms, R¹⁷ represents hydrogen, a (CH₂CH₂O)ₙR¹⁸ residue or X, h represents numbers from 1 to 10 and X represents hydrogen, an alkali or alkaline-earth metal or NR¹⁹R²⁰R²¹R²², where R¹⁹ to R²¹ represent, independently of one another, hydrogen or a C₁ to C₄ hydrocarbon residue,
sulfated fatty acid alkylene glycol esters of formula (E1-II)—

R²²CO(AIkO)ₙSO₃M (E1-II)
in which R²²CO— represents a linear or branched, aliphatic, saturated and/or unsaturated acyl residue having 6 to 22 C atoms, Alk represents CH₂CH₂, CHCH₃CH₂ and/or CH₂CHCH₃, h represents numbers from 0.5 to 5 and M represents a cation,
monoglyceride sulfates and monoglyceride ether sulfates of formula (E1-III)—

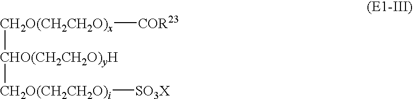

in which R²³CO represents a linear or branched acyl residue having 6 to 22 carbon atoms, x, y and i, in total, represent 0 or numbers from 1 to 30, preferably 2 to 10, and X represents an alkali or alkaline-earth metal. Typical examples of monoglyceride (ether) sulfates which are suitable in the scope of the invention are the reaction products of lauric acid monoglyceride, coco-fatty acid monoglyceride, palmitic acid monoglyceride, stearic acid monoglyceride, oleic acid monoglyceride and tallow fatty acid monoglyceride and the ethylene oxide adducts thereof with sulfur trioxide or chlorosulfonic acid in the form of the sodium salts thereof. The use of monoglyceride sulfates of formula (E1-III), in which R²³CO represents a linear acyl residue having 8 to 18 carbon atoms, is preferred,
amide ether carboxylic acids,
condensation products of C₈-C₃₀ fatty alcohols with protein hydrolyzates and/or amino acids and the derivatives thereof, which are known to the person skilled in the art as albumen fatty acid condensates such as the Lamepon®, Gluadin®, Hostapon® KCG or Amisoft® types.

Preferred anionic surfactants are alkyl sulfates, alkyl polyglycol ether sulfates and ether carboxylic acids having 10 to 18 C atoms in the alkyl group and up to 12 glycol ether groups in the molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates and albumen fatty acid condensates.

Cationic surfactants may also be used. Cationic surfactants of the quaternary ammonium compound, esterquat and amidoamine types are preferred according to the invention. Preferred quaternary ammonium compounds are ammonium halides, in particular chlorides and bromides, such as alkyl trimethyl ammonium chlorides, dialkyl dimethyl ammonium chlorides and trialkyl methyl ammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride and tricetyl methyl ammonium chloride, as well as the imidazolium compounds known by the INCI designations quaternium-27 and quaternium-83. The long alkyl chains of the aforementioned surfactants preferably have 10 to 18 carbon atoms.

Esterquats are known substances which contain both at least one ester function and at least one quaternary ammonium group as a structural element. Preferred esterquats are quaternized ester salts of fatty acids containing triethanolamine, quaternized ester salts of fatty acids containing diethanolalkylamines and quaternized ester salts of fatty acids containing 1,2-dihydroxypropyldialkylamines. Products of this type are sold for example under the brand names Stepantex®, Dehyquart® and Armocare®. Examples of esterquats of this type include the products Armocare® VGH-70, an N,N-bis(2-palmitoyloxyethyl)dimethyl ammonium chloride, as well as Dehyquart® F-75, Dehyquart® C-4046, Dehyquart® L80 and Dehyquart® AU-35.

Alkylamidoamines are conventionally prepared by amidizing natural or synthetic fatty acids and fatty acid cuts containing dialkylaminoamines. A compound of this group of substances which is particularly suitable according to the invention is stearamidopropyl dimethylamine, which is commercially available under the name Tegoamid® S 18.

Washing and cleaning agents may contain further surfactants or emulsifiers in addition to or instead of cationic surfactants, wherein anionic, ampholytic and non-ionic surfactants as well as any type of known emulsifier are suitable in principle. The group of ampholytic or else amphoteric surfactants includes zwitterionic surfactants and ampholytes. The surfactants may also have an emulsifying effect.

Surface-active compounds containing at least one quaternary ammonium group and at least one —COO⁽⁻⁾— or —SO₃⁽⁻⁾— group in the molecule are known as zwitterionic surfactants. Particularly suitable zwitterionic surfactants are betaines, such as N-alkyl-N,N-dimethyl ammonium glycinates, for example coco-alkyl dimethyl ammonium glycinate, N-acyl-aminopropyl-N,N-dimethyl ammonium glycinates, for example coco-acyl aminopropyl dimethyl ammonium glycinate, and 2-alkyl-3-carboxymethyl-3-hydroxyethyl-imidazolines, each having 8 to 18 C atoms in the alkyl or acyl group as well as coco-acyl aminoethyl hydroxy ethyl carboxymethyl glycinate. A preferred zwitterionic surfactant is the fatty acid amide derivative known by the INCI name cocamidopropyl betaine.

Ampholytes are understood to be surface-active compounds which contain at least one free amino group and at least one —COOH— or —SO₃H-group in the molecule as well as a C₈-C₂₄ alkyl or acyl group and are capable of forming inner salts. Examples of suitable ampholytes are N-alkylglycines, N-alkylpropanoic acids, N-alkylaminobutyric acids, N-alkyliminodipropanoic acids, N-hydroxyethyl-N-alkylamidopropylglycines, N-alkyltaurines, N-alkylsarcosines, 2-alkylaminopropanoic acids and alkylaminoacetic acids, each having approximately 8 to 24 C atoms in the alkyl group. Particularly preferred ampholytes are N-coco-alkylaminopropionate, coco-acylaminoethylaminopropionate and C₁₂-C₁₈ acyl sarcosine.

Non-ionic surfactants contain, for example, a polyol group, a polyalkylene glycol ether group or a combination of polyol and polyglycol ether groups as hydrophilic groups. Examples of compounds of this type include
addition products of 2 to 50 mol ethylene oxide and/or 1 to 5 mol propylene oxide to linear and branched fatty alcohols having 10 to 30 C atoms, to fatty acids having 8 to 30 C atoms and to alkylphenyls having 8 to 15 C atoms in the alkyl group,
addition products having terminal groups blocked by a methyl or a C₂-C₆ alkyl residue, of 2 to 50 mol ethylene oxide and/or 1 to 5 mol propylene oxide to linear and branched fatty alcohols having 8 to 30 C atoms, to fatty acids having 8 to 30 C atoms and to alkylphenyls having 8 to 15 C atoms in the alkyl group, such as the types available under the trade names Dehydrol® LS, Dehydrol® LT (Cognis),
C₁₂-C₃₀ fatty acid mono- and diesters of addition products of 1 to 30 mol ethylene oxide to glycerol,
addition products of 5 to 60 mol ethylene oxide to castor oil and hardened castor oil,
polyol fatty acid esters such as the commercially available Hydagen® HSP (Cognis) or Sovermol (Cognis) types,
alkoxylated triglycerides,
alkoxylated fatty acids alkyl esters of formula (E4-I)—

R²⁴CO—(OCH₂CHR²⁵)_{w}OR²⁶ (E4-I)
in which R²⁴CO represents a linear or branched, saturated and/or unsaturated acyl residue having 6 to 22 carbon atoms, R²⁵ represents hydrogen or methyl, R²⁶ represents linear or branched alkyl residues having 1 to 4 carbon atoms and w represents numbers from 1 to 20,
amine oxides,
hydroxy mixed ethers,
sorbitan fatty acid esters and addition products of ethylene oxide to sorbitan fatty acid esters such as polysorbates,
sugar fatty acid esters and addition products of ethylene oxide to sugar fatty acid esters,
addition products of ethylene oxide to fatty acid alkanolamides and fatty amines,
sugar surfactants of the alkyl and alkanyl oliglycosides according to formula (E4-II)—

R²⁷O-[G]ₚ (E4-II)
in which R²⁷ represents an alkyl or alkenyl residue having 4 to 22 carbon atoms, G represents a sugar residue having 5 or 6 carbon atoms and p represents numbers from 1 to 10. They can be obtained by the relevant methods from the field of preparative organic chemistry.
The alkyl and alkenyl oligoglycosides can be derived from aldoses or ketoses having 5 or 6 carbon atoms, preferably from glucose. The preferred alkyl and/or alkenyl oligoglycosides are thus alkyl and/or alkenyl oligoglucosides. The index number p in general formula (E4-II) denotes the degree of oligomerization (DP), that is, the distribution of mono- and oligoglycosides and is a number between 1 and 10. Whereas p is always an integer for an individual molecule and in this case may assume in particular the values p=1 to 6, the value p for a specific alkyl oligoglycoside is an analytically-determined calculated value which is generally a fraction. Alkyl and/or alkenyl oligoglycosides having an average degree of oligomerization p of 1.1 to 3.0 are preferably used. From an application perspective, alkyl and/or alkenyl oligoglycosides with a degree of oligomerization of less than 1.7 and in particular between 1.2 and 1.4 are preferred. The alkyl or alkenyl residue R²⁷ can be derived from primary alcohols having 4 to 11, preferably 8 to 10 carbon atoms. Typical examples include butanol, caproic alcohol, caprylic alcohol, capric alcohol and undecyl alcohol as well as the industrial mixtures thereof, as obtained for example by the hydrogenation of industrial fatty acid methyl esters or in the course of the hydrogenation of aldehydes in the Roelen oxosynthesis reaction. Alkyl oliglucosides with a C₈-C₁₀ chain length (DP=1 to 3) which accumulate as the first runnings in the separation, by means of distillation, of industrial C₈-C₁₈ coco fatty alcohol and which may be contaminated with a C₁₂ alcohol content of less than 6% by weight and alkyl oligoglucosides based on industrial C_{9/11} oxo alcohols (DP=1 to 3) are preferred. The alkyl or alkenyl residue R²⁷ may also be derived from primary alcohols having 12 to 22, preferably 12 to 14 carbon atoms. Typical examples include lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and the industrial mixtures thereof, which can be obtained as described above. Alkyl oligoglucosides based on hardened C_{12/14} coconut alcohol with a DP of 1 to 3 are preferred.
Sugar surfactants of the fatty acid-N-alkylpolyhydroxyalkyl amide type, a non-ionic surfactant of formula (E4-III)—

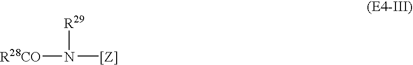

in which, R²⁸CO represents an aliphatic acyl residue having 6 to 22 carbon atoms, R²⁹ represents hydrogen, an alkyl or hydroxy alkyl residue having 1 to 4 carbon atoms and [Z] represents a linear or branched polyhydroxyalkyl residue having 3 to 12 carbon atoms and 3 to 10 hydroxyl groups. Fatty acid-N-alkylpolyhydroxyalkyl amides are known substances which can be conventionally obtained by the reductive amination of a reducing sugar with ammonia, an alkylamine or an alkanolamine and subsequent acylation with a fatty acid, a fatty acid alkyl ester or a fatty acid chloride. Fatty acid-N-alkylpolyhydroxyalkyl amides are preferably derived from reducing sugars, glucose in particular, having 5 or 6 carbon atoms. The preferred fatty acid-N-alkyl-polyhydroxyalkyl amides are therefore fatty acid-N-alkyl glucamides represented by formula (E4-IV)—

R³⁰CO—NR³¹—CH₂—(CHOH)4CH₂OH (E4-IV)
The use of glucamides of formula (E4-IV), in which R³¹ represents hydrogen or an alkyl group and R³⁰CO represents the acyl residue of caproic acid, caprylic acid, capric acid, lauric acid, myristic acid, palmitic acid, palm oleic acid, stearic acid, isostearic acid, oleic acid, elaidic acid, petroselic acid, linoleic acid, linolenic acid, arachidic acid, gadoleic acid, behenic acid or erucic acid or industrial mixtures of these acids is preferred. Fatty acid-N-alkyl glucamides of formula (E4-IV) obtained by reductive amination of glucose with methylamine and subsequent acylation using lauric acid or C_{12/14} coconut fatty acid or an appropriate derivative thereof are particularly preferred. Furthermore, the polyhydroxyalkyl amides may also be derived from maltose and palatinose.

Addition products of alkylene oxide to saturated linear fatty alcohols and fatty acids containing from 2 to 30 mol ethylene oxide being used per mol of fatty alcohol or acid, have proved to be suitable as preferred non-ionic surfactants. Preparations with excellent properties are also obtained when they contain fatty acid esters of ethoxylated glycerol as non-ionic surfactants. These compounds are characterized by the following parameters: The alkyl residue contains from 6 to 22 carbon atoms and may be linear or branched. Primary linear aliphatic residues and those with a methyl branch in position 2 are preferred. Examples of alkyl residues of this type include 1-octyl, 1-decyl, 1-lauryl, 1-myristyl, 1-cetyl and 1-stearyl. 1-octyl, 1-decyl, 1-lauryl and 1-myristyl are particularly preferred. When using “oxo alcohols” as starting materials, the predominant type of compound has an odd number of carbon atoms in the alkyl chain.

Furthermore, sugar surfactants may be contained as non-ionic surfactants. Said sugar surfactants are preferably contained in amounts of from 0.1 to 20% by weight, based on the respective total composition in the washing and cleaning agent. Amounts of from 0.5 to 15% by weight are particularly preferred and amounts of from 0.5 to 7.5% by weight are more particularly preferred.

The compounds containing alkyl groups used as surfactants may in each case be substances of a uniform length. However, it is generally preferable to use native plant or animal raw materials to prepare these substances so that substance mixtures with alkyl chains of different lengths, depending on the respective raw material used, are obtained.

Products with a “normal” homolog distribution as well as those with a narrow homolog distribution may be used as surfactants which are addition products of ethylene and/or propylene oxide to fatty alcohols or derivatives of these addition products. “Normal” homolog distribution is to be understood in this case as mixtures of homologs which are obtained when reacting fatty alcohol and alkylene oxide using alkali metals, alkali metal hydroxides or alkali metal alkoxides as catalysts. In contrast, a narrow homolog distribution is obtained when hydrotalcites, alkaline-earth metal salts of ether carboxylic acids, alkaline-earth metal oxides, hydroxides or alkoxides for example are used as catalysts. The use of products with a narrow homolog distribution range may be preferred.

The other surfactants are generally used in the washing and cleaning agents in amounts of from 0.1 to 45% by weight, preferably 0.5 to 30% by weight and more particularly preferably 0.5 to 25% by weight, based on the respective total composition. In this case, the amount used depends largely on the purpose of the particular agent. If, for example, the agent is a shampoo or another cleaning agent, surfactant amounts of greater than 45% by weight are conventional.

The surfactant content can be selected so as to be higher or lower depending on the purpose of the agent used. The surfactant content of washing agents is conventionally between 10 and 40% by weight, preferably between 12.5 and 30% by weight and in particular between 15 and 25% by weight, whereas cleaning agents for machine dishwashing for example generally contain between 0.1 and 10% by weight, preferably between 0.5 and 7.5% by weight and in particular between 1 and 5% by weight of surfactants. Soaps (toilet soaps and other soap types such as cream soaps, liquid soaps, etc.) contain between 1 and 50% by weight of surfactants depending on the type of surfactant used and the type of soap in question.

Washing and cleaning agents may further contain emulsifiers. Emulsifiers lead to the formation of water- or oil-resistant adsorbed layers, which prevent dispersed droplets from coalescing and thus stabilize the emulsion, at the phase interface. In a similar manner to surfactants, emulsifiers are therefore formed from a hydrophobic and a hydrophilic molecule part. Hydrophilic emulsifiers preferably form O/W-emulsions and hydrophilic emulsifiers preferably form W/O-emulsions. These emulsifying surfactants or emulsifiers are therefore to be selected as a function of the substances to be dispersed and the external phase and particle size of the emulsion in question. Examples of emulsifiers which may be used are:
addition products of 4 to 100 mol ethylene oxide and/or 1 to 5 propylene oxide to linear fatty alcohols having 8 to 22 C atoms, to fatty acids having 12 to 22 C atoms and to alkyl phenols having 8 to 15 C atoms in the alkyl group,
C₁₂-C₂₂ fatty acid mono- and diesters of addition products of 1 to 30 mol ethylene oxide to polyols having 3 to 6 carbon atoms, in particular glycerol,
addition products of ethylene oxide and polyglycerol to methyl glucoside fatty acid esters, fatty acid alkanolamides and fatty acid glucamides,
C₈-C₂₂ alkyl mono- and oligoglycosides and the ethoxylated analogs thereof, wherein the degree of oligomerization is preferably of from 1.1 to 5, in particular 1.2 to 2.0, and glucose is preferably used as the sugar component,
mixtures of alkyl (oligo)glucosides and fatty alcohols, for example the commercially available product Montanov® 68,
addition products of 5 to 60 mol ethylene oxide to castor oil and hardened castor oil,
partial esters of polyols having 3 to 6 carbon atoms with saturated fatty acids having 8 to 22 C atoms,
sterols. Sterols are understood to be a group of steroids which have a hydroxyl group at C atom 3 of the steroid skeleton and are isolated both from animal tissue (zoosterols) and from vegetable fats (phytosterols). Examples of zoosterols are cholesterol and lanosterol. Examples of suitable phytosterols include ergosterol, stigmasterol and cytosterol. Sterols, such as mycosterols as they are known, may also be isolated from fungi and yeasts.
phospholipids. This category is to be understood as encompassing in particular glucose phopholipids which are obtained for example as lecithins or phosphatidyl cholines, for example from egg yolk or plant seeds (soybeans for instance).
fatty acid esters of sugars and sugar alcohols such as sorbitol,
polyglycerols and polyglycerol derivatives such as polyglycerol poly-12-hydroxystearate (commercial product Dehymuls® PGPH),
linear and branched fatty acids having 8 to 30 C atoms and the Na, K, ammonium, Ca, Mg and Zn salts thereof.

Emulsifiers are preferably used in amounts of from 0.1 to 25% by weight, in particular 0.1 to 3% by weight, based on the total amount of the composition in question.

Builders are another important group of ingredients of washing and cleaning agents. This category includes both organic and inorganic builder substances. Builders are compounds which may carry out a supporting function in the agents and also act as a water softener when in use.

Examples of suitable builders include alkali metal gluconates, citrates, nitrilotriactetates, carbonates and bicarbonates, in particular sodium gluconate, citronate and nitrilotriactetate as well as sodium and potassium carbonate and bicarbonate, and alkali metal and alkaline-earth metal hydroxides, in particular sodium and potassium hydroxide, ammonia and amines, in particular mono- and triethanolamine, and the mixtures thereof. The salts of glutaric acid, succinic acid, adipic acid, tartaric acid and benzene hexacarboxylic acid as well as phosphonates and phosphates are included in this category.

Employable organic builder substances include for example polycarboxylic acids in the form of the sodium salts thereof, the term polycarboxylic acids being understood as meaning carboxylic acids which have more than one acid function. This category includes, for example, citric acid, adipic acid, succinic acid, glutaric acid, malic acid, tartaric acid, maleic acid, fumaric acid, saccharic acids, aminocarboxylic acids, nitrilotriacetic acid (NTA), provided that the use thereof is not ecologically unacceptable, and mixtures thereof. Preferred salts are the salts of polycarboxylic acids such as citric acid, adipic acid, succinic acid, glutaric acid, tartaric acid, saccharic acids and mixtures thereof. The acids themselves may also be used per se. In addition to their builder effect, the acids also typically exhibit the characteristics of an acidification component and thus also cause the washing or cleaning agents to have a lower and milder pH, such as in granulates according to the invention. Particularly preferred acids are in this case citric acid, succinic acid, glutaric acid, adipic acid, gluconic acid and any desired mixtures thereof.

Further substances suitable for use as builders are polymeric polycarboxylates. These are, for example, the alkali metal salts of polyacrylic acid or polymethacrylic acid, having a relative molecular mass of from 500 to 70,000 g/mol for instance. The (co)polymeric polycarboxylates may be used in the form of either a powder or an aqueous solution. The content of (co)polymeric polycarboxylates in the agent is preferably of from 0.5 to 20% by weight, in particular of form 3 to 10% by weight. The polymers may also contain allyl sulfonic acids, allyl oxybenzene sulfonic acid and methallyl sulfonic acid in the form of monomers, in order to improve the solubility in water. Particularly preferred polymers are biologically degradable polymers formed from more than two different monomer units such as polymers containing acrylic acid and maleic acid salts and vinyl alcohol or vinyl alcohol derivatives as monomers, or acrylic acid and 2-alkylallyl sulfonic acid salts and sugar derivatives as monomers. Further preferred copolymers preferably contain acrolein and acrylic acid/acrylic acid salts or acrolein and vinyl acetate as monomers. Similarly, further preferred builder substances include polymeric amino dicarboxylic acids, the salts or precursors thereof. Polyaspartic acids or the salts and derivatives thereof are particularly preferred as they have both cobuilder properties and a bleach-stabilizing effect.

Further suitable builder substances are polyoxymethylenes which are obtained by reacting dialdehydes with polyol carboxylic acids having 5 to 7 C atoms and at least 3 hydroxyl groups. Preferred polyoxymethylenes are obtained from dialdehydes such as glyoxal, glutaraldehyde, terephthaldehyde and the mixtures thereof and from polyol carboxylic acids such as gluconic acid and/or glucoheptonic acid.

Dextrins such as carbohydrate oligomers or polymers which can be obtained via the partial hydrolysis of starch are further suitable organic builders. The hydrolysis reaction may be carried out in accordance with conventional methods, for example may be catalyzed by acids or enzymes. The dextrins are preferably hydrolysis products with an average molar mass in the range of from 400 to 500,000 g/mol. In this case, a polysaccharide with a dextrose equivalent (DE) value in the range of from 0.5 to 40, in particular 2 to 30, is preferred, wherein DE is a widely used measure of the reducing action of a polysaccharide in comparison with dextrose, which has a DE of 100. Maltodextrins with a DE value of between 3 and 20 and dried glucose syrup with a DE value of between 20 and 37, as well as what are known as yellow and white dextrins which have higher molar masses in the range of from 2,000 to 30,000 g/mol may be used. A preferred dextrin is described in British patent application 94 19 091. Oxidized derivatives of dextrins of this type are the reaction products thereof with oxidation agents which are capable of oxidizing at least one alcohol function of the saccharide ring to form a carboxylic acid function.

Oxydisuccinates and other disuccinate derivatives, preferably ethylene diamine succinate, are also further suitable cobuilders. Ethylene diamine-N—N′-disuccinate (EDDS), the synthesis of which is described for example in U.S. Pat. No. 3,158,615, is in this case preferably used in the form of the sodium or magnesium salts thereof. Glycerol disuccinates and glycerol trisuccinates are also preferred. Suitable quantities for use in zeolite-containing and/or silicate-containing formulations are of from 3 to 15% by weight.

Further examples of employable organic cobuilders are acetylated hydroxycarboxylic acids or the salts thereof which may also optionally be present in lactone form and contain at least 4 carbon atoms and at least one hydroxyl group in addition to a maximum of two acid groups.

A further category of substances having cobuilder properties are the phosphonates, in particular hydroxyalkane and aminoalkane phosphonates. Of the substances in the hydroxylalkane phosphonate category, 1-hydroxyethane-1,1-diphosphonate (HEDP) is of particular importance as a cobuilder. It is preferably employed in form of a sodium salt, wherein the sodium salt reacts neutral and the tetrasodium salt reacts alkaline (pH 9). Ethylene diamine tetramethylene phosphonate (EDTMP), diethylenetriamine pentamethylene phosphonate (DTPMP) and the higher homologs thereof may preferably be used as aminoalkane phosphonates. They are preferably used in the form of the neutral-reacting sodium salts, for example the hexasodium salt of EDTMP or the hepta- and octasodium salt of DTPMP. Of the substances in the phosphonate category, HEDP is preferably used as a builder in this case. Aminoalkane phosphonates also exhibit a pronounced heavy-metal-binding power. It may consequently be preferable to use aminoalkane phosphonates, in particular DTPMP, or mixtures of the aforementioned phosphonates, in particular if the agents also contain bleach.

Furthermore, any compounds which are capable of forming complexes with alkaline-earth ions may be used as cobuilders.

A fine-grained, synthetic zeolite containing bound water is preferably used as an inorganic builder. The fine grained, synthetic bound-water-containing zeolite used is preferably zeolite A and/or P. For example, Zeolite MAP, for example Doucil A24® (commercial product sold by Crosfield), may be used as zeolite P. However, zeolite X and mixtures of A, X and/or P, for example a co-crystallizate formed from zeolites A and X, Vegobond® AX (commercial product sold by Condea Augusta S.p.A.), are also suitable. The zeolite may be used as a spray-dried powder or as a non-dried stabilized suspension which is still moist from its preparation process. If zeolite is used in the form of a suspension, it may contain small additional amounts of non-ionic surfactants as stabilizers, for example 1 to 3% by weight, based on the zeolite, of ethoxylated C₁₂-C₁₈ fatty alcohols having 2 to 5 ethylene oxide groups, C₁₂-C₁₄ fatty alcohols having 4 to 5 ethylene oxide groups or ethoxylated isotridecanols. Suitable zeolites have a mean particle size of less than 10 μm (volume distribution; measurement method: Coulter counter) and preferably contain 18 to 22% by weight, in particular 20 to 22% by weight of bound water. In preferred embodiments, the zeolite content in the premix is of from 10 to 94.5% by weight, wherein it may be particularly preferable for the zeolite content to be of from 20 to 70, in particular 30 to 60% by weight.

Suitable partial substitutes for zeolites are phyllosilicates of natural and synthetic origin. They may have any desired composition or structural formula, but smectites, and in particular bentonite, are preferred. Crystalline, layered sodium silicates of general formula NaMSiₓO₂ₓ₊₁.yH₂O, wherein M is sodium or hydrogen, x is a number from 1.9 to 4 and y is a number from 0 to 20 and preferred values for x are 2, 3 or 4, are also suitable as zeolite or phosphate substitutes. Preferred crystalline phyllosilicates of the aforementioned formula are those in which M represents sodium and x assumes the values 2 or 3. Both β- and δ-sodium disilicates Na₂Si₂O₅.yH₂O are particularly preferred.

It is obviously possible to use the generally known phosphates as builder substances, provided that the use thereof is not to be avoided on ecological grounds. Sodium salts of orthophosphates, pyrophosphates and in particular tripolyphosphates are particularly suitable.

Builders are preferably used in amounts of from 0 to 20% by weight, preferably 0.01 to 12% by weight, in particular 0.1 to 8% by weight and most preferably 0.3 to 5% by weight, based on weight of the composition.

In addition to the aforementioned components, washing and cleaning agents may also contain one or more substances from the group of bleaching agents, bleach activators, enzymes, pH-adjusting agents, fluorescing agents, dyes, suds suppressors, silicone oils, anti-redeposition agents, optical brighteners, graying inhibitors, dye transfer inhibitors, corrosion inhibitors and silver protection agents. These substances will be described below.

From the group of compounds which act as bleaching agents and yield H₂O₂ in water, sodium perborate tetrahydrate, sodium perborate monohydrate and sodium percarbonate are of particular significance. Further examples of bleaching agents which may be used are peroxypyrophosphates, citrate perhydrates as well as H₂O₂-yielding peracid salts or peracids, such as perbenzoates, peroxophthalates, diperazelaic acid, phthaloiminoperacid or diperdodecane diacid. If bleaching agents are used, it is also possible to dispense with surfactants and/or builders so pure bleaching agent tablets may be produced. If bleaching agent tablets of this type are used for washing textiles, it is preferable to use a combination of sodium percarbonate and sodium sesquicarbonate, irrespective of the other ingredients of the shaped bodies. If cleaning or bleaching agent tablets for dishwashers are produced then it is also possible to use bleaching agents from the group of organic bleaching agents. Typical organic bleaching agents are diacyl peroxides such as dibenzoyl peroxide. Further typical organic bleaching agents are peroxy acids, wherein examples thereof include alkylperoxy acids and arylperoxy acids. Preferred representatives of this category are (a) peroxybenzoic acid and the ring-substituted derivatives thereof such as alkyl peroxy benzoic acids, but also peroxy-α-naphthoic acid and magnesium monoperphthalate, (b) aliphatic or substituted aliphatic peroxy acids, such as peroxylauric acid, peroxystearic acid, ε-phthalimidoperoxycaproic acid [phthaloiminoperoxyhexanoic acid (PAP)], o-carboxybenzamidoperoxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-piperoxyazelaic acid, diperoxysebacic acid, diperoxybrassylic acid, diperoxyphthalic acids, 2-decyldiperoxybutane-1,4-dioic acid, N,N-terephthaloyl-di(6-aminopercaproic acid) may also be used. Bleach activators may be incorporated into the washing and cleaning agent according to the invention to obtain an improved bleaching effect when washing or cleaning at temperatures of 60° C. and less. Compounds which produce aliphatic peroxocarboxylic acids preferably having 1 to 10 C atoms, in particular 2 to 4 C atoms, and/or optionally substituted perbenzoic acid under perhydrolysis conditions may be used as bleach activators. Substances having O- and/or N-acyl groups with the aforementioned number of C atoms and/or optionally substituted benzoyl groups are suitable. Polyacylated alkylendiamines, in particular tetraacetylethylenediamine (TAED), acylated triazine derivates, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenol sulfonates, in particular n-nonanoyl- or isononanoyl oxybenzene sulfonate (n- or iso-NOBS), carboxylic acid anhydrides, in particular phthalic acid anhydride, acylated polyhydric alcohols, in particular triacetin, ethylene glycol diacetate and 2,5-diacetoxy-2,5-dihydrofuran are preferred.

What are known as bleach catalysts may be used in addition to or instead of conventional bleach activators. These substances are bleach-boosting transition metal salts or transition metal complexes such as Mn-, Fe-, Co-, Ru- or Mo-Salen or -carbonyl complexes. Mn-, Fe-, Co-, Ru-, Mo-, Ti-, V- and Cu-complexes with N-containing tripod ligands as well as Co-, Fe-, Cu- and Ru-ammine complexes may also be used as bleach catalysts.

Enzymes from the groups of proteases, lipases, amylases, cellulases and mixtures thereof are suitable for use as enzymes. Enzymatic active ingredients obtained from strains of bacteria or fungi such as Bacillus subtilis, Bacillus licheniformis and Streptomyces griseus are particularly suitable. Subtilisin-type proteases, in particular proteases obtained from Bacillus lentus, are preferably used. In this case, enzyme mixtures, for example protease and amylase or protease and lipase or protease and cellulase or of cellulase and lipase or of protease, amylase and lipase or protease, lipase and cellulase, and in particular cellulase-containing mixtures, are of particular benefit. Peroxidases and oxidases have also proved to be suitable in some cases. The enzymes may be adsorbed to substrates and/or embedded in coating substances to prevent them from decomposing prematurely. The enzyme content, enzyme mixture content or enzyme granulate content in the shaped bodies according to the invention may for example be of from approximately 0.1 to 5% by weight, preferably 0.1 to approximately 2% by weight. The most frequently used enzymes are lipases, amylases, cellulases and proteases. Preferred proteases are BLAP®140 from Biozym, Optimase®-M-440 and Opticlean®-M-250 from Solvay Enzymes; Maxacal®CX and Maxapem® or Esperase® from Gist Brocades or also Savinase® from Novo. Particularly suitable cellulases and lipases are Celluzyme® 0.7 T and Lipolase® 30 T from Novo Nordisk. Duramyl® and Termamyl® 60 T, and Termamyl® 90 T from Novo, Amylase-LT® from Solvay Enzymes or Maxamyl® P5000 from Gist Brocades are to be used in particular as amylases. Other enzymes may also be used.

The washing and cleaning agents may also contain components which make it easier to wash oil and grease out of textiles (what are known as soil repellants). This effect is particularly pronounced when a textile which has previously been washed a number of times with a washing agent according to the invention which contains these oil- and grease-dissolving components, is washed. Examples of the preferred oil- and grease-dissolving components include non-ionic cellulose ethers such as methylcellulose and methylhydroxypropylcellulose containing 15 to 30% by weight of methoxyl groups and 1 to 15% by weight of hydroxylpropoxyl groups, in each case based on the non-ionic cellulose ethers, and known polymers of phthalic acid and/or terephthalic acid or the derivatives thereof, in particular ethylene terephthalate and/or polyethylene glycol terephthalate polymers or anionically and/or non-ionically modified derivatives thereof. The particularly preferred substances in this category are sulfonated derivatives of phthalic acid and/or terephthalic acid polymers.

The agents may contain derivatives of diaminostilbene disulfonic acid or the alkali metal salts thereof as optical brighteners. Salts of 4,4′-bis(2-anilino-4-morpholino-1,3,5-triazinyl-6-amino)stilbene-2,2′-disulfonic acid or similarly constructed compounds with a diethanolamino group, a methylamino group, an anilino group or a 2-methoxyethylamino group instead of the morpholino group are suitable for example. Furthermore, brighteners of the substituted diphenol styryl type may also be present, for example the alkali salts of 4,4′-bis(2-sulfostyryl)diphenyl, 4,4′-bis(4-chloro-3-sulfos Dbiphenyl, or 4-(4-chlorostyryl)-4′-(2-sulfostyryl)diphenyl. Mixtures of the aforementioned brighteners may also be used.

The agents may be dyed using suitable dyes in order to improve their appearance. Preferred dyes, which can be selected by the person skilled in the art without difficulty, are stable in storage and are not affected by the other ingredients of the agent or light, and do not exhibit pronounced substantivity in relation to textile fibers in order to avoid dyeing said fibers.

This list of ingredients of washing and cleaning agents is by no means exhaustive and only provides the main typical ingredients of agents of this type. In particular, the agents may also contain organic solvents if they are liquid or gel preparations. These solvents are preferably monohydric or polyhydric alcohols having 1 to 4 C atoms. The alcohols contained in agents of this type are preferably ethanol, 1,2-propanediol, glycerol and mixtures of these alcohols. In preferred embodiments, agents of this type contain 2 to 12% by weight of these alcohols.

In principle, the agents may be in different states of aggregation. In a further preferred embodiment, the soaps are solid, gel or pasty soaps, wherein solid soaps are preferred.

In a further preferred embodiment, the washing or cleaning agents are liquid or gel agents, in particular liquid washing agents or liquid dishwashing agents or cleaning gels, wherein they may also be gel cleaning agents for flushing toilets in particular. In this case they are preferably gel pseudoplastic cleaning agents which have a viscosity of from 30,000 to 150,000 mPas and contain a polysaccharide as a gel former, a C₈₋₁₀ alkyl polyglycoside or C₁₂₋₁₄ alkyl polyglycoside as an emulsifier and wetting component, and perfume oil. Fatty alcohol ether sulfates (FAEOS) and fatty alcohol sulfates (FAS) may be contained as additional co-surfactants. In this case, the APG/co-surfactant ratio is generally greater than 1, preferably between 50:1 and 1:1, particularly preferably between 10:1 and 1.5:1 and more particularly preferably between 5:1 and 1.8:1. In this case, these agents are in particular stable, shear-thinning gel cleaning agents which contain polysaccharide, a surfactant system and perfume components and are characterized in that
they contain a polysaccharide, preferably a xanthan gum, in an amount of between 1 and 5% by weight, preferably of from 1 to 4% by weight, particularly preferably of from 1.5 to 3.5% by weight and more particularly preferably of from 1.8 to 3% by weight,
a C₈₋₂₂ alkyl polyglycoside as a component of the surfactant system in an amount of between 3 and 25% by weight, preferably 4 and 20% by weight, particularly preferably 5 and 15% by weight and more particularly preferably 5 and 12% by weight, and
the perfume component or components in an amount of up to 15% by weight, preferably between 2 and 12% by weight, particularly preferably between 3 and 8% by weight,
as well as further optional ingredients such as limescale-dissolving agents, dyes, bacteriostatic agents (such as isothiazoline mixtures, sodium benzoate or salicylic acid), pearlescing agents, stabilizers, cleaning boosters and odor absorbers,
and they have a viscosity of 30,000 to 150,000 mPas, measured using a Brookfield rotation viscometer of the RVT type with a helipath stand and spindle TA at 1 rpm and 23° C.

The gels according to the invention may optionally contain water-soluble and water-insoluble builders. In this case, water-soluble builders are preferred since they are generally less likely to form insoluble residues on hard surfaces. Conventional builders which may be added within the scope of the invention are low-molecular polycarboxylic acids and the salts thereof, homopolymeric and copolymeric polycarboxylic acids and the salts thereof, citric acid and the salts thereof, carbonates phosphates and silicates. The category of water-insoluble builders includes zeolites, which may also be used, as well as mixtures of the aforementioned builder substances. The citrate group is particularly preferred.

In a particularly advantageous embodiment, the aforementioned agents may contain one or more hydrophobic components. Examples of suitable hydrophobic components are dialkyl ethers having the same or different C₄₋₁₄ alkyl residues, in particular dioctyl ether; hydrocarbons with a boiling point range of from 100 to 300° C., in particular of from 140 to 280° C., for example aliphatic hydrocarbons with a boiling point range of from 145 to 200° C. and isoparaffins with a boiling point range of from 200 to 260° C.; essential oils, in particular limonene and pine oil extracted from pine roots and stumps; and also mixtures of these hydrophobic components, in particular mixtures of two or three of the aforementioned hydrophobic components. Preferred mixtures of hydrophobic components are mixtures of different dialkyl ethers, of dialkyl ethers and hydrocarbons, of dialkyl ethers and essential oils, of carbohydrates and essential oils, of dialkyl ethers and hydrocarbons and essential oils and of these mixtures. The agents have a hydrophobic component content of from 0 to 20% by weight, preferably 0.1 to 14% by weight, in particular 0.5 to 10% by weight, and extremely preferably 0.8 to 7% by weight, based on the composition.

All-purpose cleaners may also contain soaps, that is, the alkali or ammonium salts of saturated or unsaturated C₆₋₂₂ fatty acids, on account of the suds suppressing properties thereof. The soaps may be used in an amount of up to 5% by weight, preferably of from 0.1 to 2% by weight.

In addition to the aforementioned components, washing and cleaning agents may contain further auxiliaries and additives conventionally used in agents of this type. These include, in particular, polymers, soil-release active ingredients, solvents (for example ethanol, isopropanol, glycol ether), solubilizers, hydrotropic substances (such as cumene sulfonate, octyl sulfate, butyl glucoside, butyl glycol), cleaning boosters, viscosity regulators (for example synthetic polymers such as polysaccharides, polyacrylates, polymers and the derivatives thereof present in nature such as xanthan gum, other polysaccharides and/or gelatins), pH regulators (such as citric acid, alkanolamines or NaOH), disinfectants, antistatic agents, preservatives, bleach systems, enzymes, dyes as well as opacifying agents or skin protection agents. The amount of additives of this type in a cleaning agent is usually no greater than 12% by weight. The lower limit depends on the additive type and may for example be as low as 0.001% by weight or less for dyes. The auxiliary content is preferably from 0.01 to 7% by weight, in particular, from 0.1 to 4% by weight.

The aforementioned agents may further comprise binders which may be used alone or mixed with other binders. Preferred binders are polyethylene glycols, 1,2-polypropylene glycols as well as modified polyethylene glycols and polypropylene glycols. The group of modified polyalkylene glycols includes in particular the sulfates and/or disulfates of polyethylene glycols or polypropylene glycols having a relative molecular mass of from 600 to 12,000, in particular from 1,000 to 4,000. A further group consists of polyalkylene glycol mono- and/or disuccinates having relative molecular masses of from 600 to 6,000, preferably from 1,000 to 4,000. In the scope of the present invention, the polyethylene glycols include polymers which have been produced using C₃-C₅ glycols, glycerol and mixtures thereof as well as ethylene glycol, as primers. In addition, ethoxylated derivatives such as trimethylol propane with 5 to 30 ethylene oxide (EO) are also included. The preferred polyethylene glycols may have a linear or branched structure, wherein linear polyethylene glycols are particularly preferred. The category of particularly preferred polyethylene glycols includes those with relative molecular masses of from 2,000 to 12,000, advantageously approximately 4,000, wherein polyethylene glycols of less than 3,500 and more than 5,000 can be used in particular in a combination with polyethylene glycols with a relative molecular mass of approximately 4,000, and wherein combinations of this type advantageously comprise more than 50% by weight, based on the total weight of the polyethylene glycols, of polyethylene glycols with a relative molecular mass of from 3,500 to 5,000. However, it is also possible to use polyethylene glycols which are in a liquid state at ambient temperature and at a pressure of 1 bar, as binders, reference in this case predominantly being made to polyethylene glycol with a relative molecular mass of 200, 400 and 600. However, these inherently liquid polyethylene glycols should only be used in a mixture with at least one further binder, wherein this mixture must again meet the requirements according to the invention, that is to say have a melting or softening point of at least greater than 45° C.

Low-molecular polyvinyl pyrrolidones and derivatives thereof with relative molecular masses of up to 30,000 are also suitable as binders. In this case, relative molecular mass ranges of from 3,000 to 30,000, for example approximately 10,000, are preferred. Polyvinyl pyrrolidones are preferably used in combination with other binders, in particular in combination with polyethylene glycols, rather than being used as the sole binder.

Raw materials which have a washing or cleaning effect, that is, non-ionic surfactants with a melting point of at least 45° C. or mixtures of non-ionic surfactants and other binders for example, have also proved suitable as binders. Preferred non-ionic surfactants include alkoxylated fatty or oxo alcohols, in particular C₁₂₋₁₈ alcohols. In this case, degrees of alkoxylation, in particular ethyloxylation, of an average of 18 to 80 AO (alkylene oxide) units, in particular ethylene oxide (EO) units per mol alcohol and mixtures thereof have proved to be advantageous. In particular, fatty alcohols with an average of 18 to 35 EO units, in particular an average of 20 to 25 EO units, exhibit advantageous binder properties in the meaning of the present invention. Ethoxylated alcohols with a lower average number of EO units per mol of alcohol, such as tallow fatty alcohol with 14 EO units, may also optionally be contained in binder mixtures. However, these relatively low-ethoxylated alcohols are preferably only used in a mixture with more highly ethoxylated alcohols. The content of these relatively low-ethoxylated alcohols in the binder is advantageously less than 50% by weight, in particular less than 40% by weight, based on the total amount of binder used. In particular, non-ionic surfactants such as C₁₂₋₁₈ alcohols with an average of 3 to 7 EO units, which are conventionally used in washing or cleaning agents and are liquid per se at ambient temperature, are preferably contained in the binder mixtures only in such an amount that the end product of the process contains less than 2% of these non-ionic surfactants. As mentioned above, the use of non-ionic surfactants which are liquid at ambient temperature in the binder mixtures is less preferable. However, in a particularly advantageous embodiment, non-ionic surfactants of this type are not a component of the binder mixture, since they not only lower the softening point of the mixture but may also increase the adhesiveness of the final product and, in addition, do not sufficiently satisfy the requirement of rapid dissolution of the binder/partition wall in the final product on account of its tendency to cause gelling upon contact with water. It is also not preferable for anionic surfactants or the precursors thereof, anionic surfactant acids, which are conventionally used in washing or cleaning agents, to be contained in the binder mixture. Other non-ionic surfactants which are suitable for use as binders are fatty acid methyl ester ethoxylates which do not tend to cause gelling, in particular those with an average of 10 to 25 EO units (see below for a more detailed description of this group of substances). Particularly preferred examples of this group of substances are methyl esters based primarily on C₁₆₋₁₈ fatty acids, for example hardened beef tallow methyl esters with an average of 12 EO units or an average of 20 EO units. In a preferred embodiment of the invention a coconut- or tallow-based C₁₂₋₁₈ fatty alcohol with an average of 20 EO units and polyethylene glycol with a relative molecular mass of from 400 to 4,000 is used as a binder. In another preferred embodiment of the invention, a mixture containing methyl esters, based predominantly on C₁₆₋₁₈ fatty acids, with an average of 10 to 25 EO units, in particular hardened beef tallow methyl esters with an average of 12 EO units or an average of 20 EO units, and a coconut- or tallow-based C₁₂₋₁₈ fatty alcohol with an average of 20 EO units and/or polyethylene glycol with a relative molecular mass of from 400 to 4,000 are used.

Binders based either solely on polyethylene glycols with a relative molecular mass of approximately 4,000 or on a mixture of coconut- or tallow-based C₁₂₋₁₈ fatty alcohol with an average of 20 EO and one of the fatty acid methyl ester ethoxylates described above or on a mixture of coconut- or tallow-based C₁₂₋₁₈ fatty alcohol with an average of 20 EO, one of the fatty acid methyl ester ethoxylates described above and a polyethylene glycol, in particular with a relative molecular mass of 4,000, have proved to be particularly advantageous embodiments of the invention.

The agent according to the invention may contain carbonate/citric acid-systems for example as suitable, well-known disintegration agents, wherein other organic acids may also be used. Swelling disintegration agents include for example synthetic polymers such as polyvinylpyrrolidone (PVP) or natural polymers or modified natural products such as cellulose and starch and the derivatives thereof, alginates or casein derivatives.

In the scope of the present invention, cellulose-based disintegration agents are used as preferred disintegration agents, so preferred washing and cleaning agent shaped bodies contain a cellulose-based disintegration agent of this type in an amount of from 0.5 to 10% by weight, preferably 3 to 7% by weight and in particular 4 to 6% by weight. Pure cellulose has the formal gross composition (C₆H₁₀O₅)ₙ and is formally considered to be a β-1,4-polyoxymethylene of cellubiose, which itself is composed of two glucose molecules. In this case, suitable celluloses are composed of from approximately 500 to 5,000 glucose units and therefore have an average molecular mass of from 50,000 to 500,000. Cellulose derivatives which can be obtained from cellulose by polymer-like reactions may also be used as cellulose-based disintegration agents in the scope of the present invention. Chemically modified celluloses of this type in this case encompass products of esterification or etherification reactions in which hydroxy hydrogen atoms have been substituted. However, celluloses in which the hydroxy groups have been replaced by functional groups which are not bound by an acid atom may also be used as cellulose derivatives. The group of cellulose derivatives includes, for example, alkali celluloses, carboxymethyl cellulose (CMC), cellulose esters and ethers as well as amino celluloses. The aforementioned cellulose derivatives are preferably mixed with cellulose rather than being used alone as cellulose-based disintegration agents. The cellulose derivative content in these mixtures is preferably less than 50% by weight, particularly preferably less than 20% by weight, based on the cellulose-based disintegration agent. Pure cellulose free of cellulose derivatives is particularly preferably used as a cellulose-based disintegration agent.

The cellulose used as a disintegration aid is preferably not used in fine-particle form but is rather converted into a coarser form, granulated or compacted for example, before being added to the premixes to be compressed. The particle size of disintegration agents of this type is generally greater than 200 μm, preferably between 300 and 1,600 μm for up to at least 90% thereof, and in particular between 400 and 1,200 μm for up to at least 90% thereof.

Microcrystalline cellulose can be used as a further cellulose-based disintegration agent or as a constituent of this component. Microcrystalline cellulose is obtained by the partial hydrolysis of cellulose under conditions which affect and completely dissolve only the amorphous regions (approximately 30% of the total mass of the cellulose) of the cellulose but leave the crystalline regions thereof (approximately 70%) untouched. Subsequent disaggregation of the micro-fine celluloses produced by the hydrolysis reaction provides microcrystalline celluloses which have a primary particle size of approximately 5 μm and can be compacted, for example to form granulates having an average particle size of 200 μm.

In a preferred variant, washing and cleaning agents, in particular in the form of shaped bodies such as tablets, contain 0.5 to 10% by weight, preferably 3 to 7% by weight, and in particular, 4 to 6% by weight of one or more disintegration auxiliaries, based on the weight of the shaped body in each case.

In a preferred embodiment, the (calcium, potassium, sodium) iodides are used in combination with vanillin and/or vanillin derivatives (according to formula (I)), in cosmetic agents for hair or skincare, for example skin creams, skin lotions, skin oils, gels and soaps, as well as hair conditioners, hair gels, intense hair conditioning treatments, hair creams, hair lotions and shampoos.

In a further preferred embodiment, the cosmetic agents are aqueous preparations which contain surface-active substances and are suitable in particular for the care of keratin fibers, in particular, human hair, or skincare.

The aforementioned hair care agents are in this case agents for the care of human head hair in particular. The most common agents of this group are categorized into hair washing agents, hair care agents, hair setting and styling agents, hair dyes and hair removal agents. The group of agents which are preferred according to the invention and contain surface-active substances include hair washing and hair care agents in particular. A hair washing agent or shampoo of this type consists of from 10 to 20, in some cases up to 30 formulation components. These aqueous preparations are predominantly in liquid to pasty form. The aforementioned cosmetic agents also generally contain further ingredients which are conventionally used for agents of this type.

The cosmetic agents preferably contain surface-active substances or substances with a washing effect as further ingredients. In this case, fatty alcohol polyglycol ether sulfates (ether sulfates, alkyl ether sulfates) are preferably used, partly in combination with other generally anionic surfactants. Besides alkyl ether sulfates, preferred agents may also contain further surfactants such as alkyl sulfates, alkyl ether carboxylates, preferably with degrees of ethoxylation of from 4 to 10, and surfactant albumen fatty acid concentrates. In this respect albumen abietic acid condensate is to be mentioned in particular. Sulfosuccinic acid esters, amidopropyl betaines, amphoacetates, amphodiacetates and alkyl polyglycosides are also surfactants which are preferably used in hair shampoos.

A further group of ingredients are subsumed under the term auxiliaries and includes a wide range of substances: for example, the addition of non-ionic surfactants, such as ethoxylated sorbitan esters, or of albumen hydrolyzates increase compatibility with the skin or minimize irritation, in baby shampoos for example; natural oils or synthetic fatty acid esters for example act as lipid replenishers to prevent excessive drying when washing hair; glycerol, sorbitol, propylene glycol (see propane diols), polyethylene glycols, including polyols, act as moisture retention agents. Cationic surfactants such as quaternary ammonium compounds may be added to the shampoos to make the hair easier to comb when wet and to reduce the accumulation of electrostatic charge in the hair after drying. Dyes or pearlescing pigments are added for a colored, glossy appearance. Thickening agents of different substance categories may be used to achieve the desired viscosity and pH stability is obtained by the use of citrate-, lactate- or phosphate-based buffers for example. Preservatives such as 4-hydroxybenzoic acid esters are added to ensure a sufficient shelf and storage life. Ingredients susceptible to oxidation may be protected by adding antioxidants, such as ascorbic acid, butylmethoxyphenyl or tocopherol.

A further preferred group of ingredients comprises specific active ingredients for specific-purpose shampoos, for example oils, herb extracts, proteins, vitamins and lecithins in shampoos for greasy, particularly dry, stressed or damaged hair. Active ingredients in anti-dandruff shampoos generally have a broad growth-inhibiting effect against fungi and bacteria. In particular, an effective anti-dandruff action has been found to be provided by substances, pyrithione salts for example, which exhibit good fungistatic properties. Hair shampoos contain perfume oils to produce a pleasant odor. Any conventional fragrances authorized for use in hair shampoos may be used in this case.

The purpose of hair care agents is to maintain the natural state of newly grown hair for as long as possible and to restore damaged hair to this state. Features which characterize this natural state are a silky shine, low porosity, resilient and thus soft body and a pleasant smooth feel. An important requirement for this is a clean, dandruff-free and not overly greasy scalp. Nowadays, the range of hair care agents includes a large number of different products, the main ones being pre-treatment agents, hair tonics, styling aids, hair conditioners, and hair repair kits, the composition of which can, in a similar manner to the hair washing agents, be divided roughly into basic substances, auxiliaries and specific active ingredients.

The group of basic substances includes fatty alcohols, in particular cetyl alcohol (1-hexadecanol) and stearyl alcohol (1-octodecanol), waxes such as beeswax, wool wax (lanolin), spermaceti wax and synthetic waxes, paraffins, vaselines and paraffin oil, and ethanol, 2-propanol and water in particular as solvents. Auxiliaries are emulsifiers, thickening agents, preservatives, antioxidants, dyes and perfume oils. Nowadays, quaternary ammonium compounds are the most important group of specific active ingredients used in hair care agents. A distinction is made between monomeric (for example: alkyltrimethylammonium halide having a lauryl, cetyl or stearyl group in particular as the alkyl residue) and polymeric quaternary ammonium compounds [for example: quaternary cellulose ether derivatives or poIy(N,N-dimethyl-3,4-methylenepyrrolidinium chloride)]. These compounds have such an effect in hair care agents since the positive charge of the nitrogen atoms in these compounds is able to attach itself to the negative charges of the keratin in the hair. Damaged hair contains more negatively charged acid groups due to the higher cysteinic acid content thereof and is therefore able to absorb more quaternary ammonium compounds. These compounds, which are also termed “cationic hair care substances” on account of the cationic nature thereof, have a smoothing effect on the hair, enable it to be combed more easily, reduce electrostatic charge and improve hold and shine. The polymeric quaternary ammonium compounds adhere to hair so well that the effect thereof can still be detected after several washes. Organic acids such as citric acid, tartaric acid or lactic acid are frequently used to obtain an acidic medium. Water-soluble albumen hydrolizates attach well to the keratin of the hair owing to the close chemical affinity thereof.

The largest group of specific active ingredients in hair care agents is formed by various plant extracts and oils.

These extracts are conventionally prepared by being extracted from the entire plant. In some cases, it may also be preferable to obtain the extracts exclusively from the blossom and/or leaves of the plant. In relation to the plant extracts preferred according to the invention, reference is made in particular to the extracts listed in the table beginning on page 44 of the Guide for Declaration of Ingredients of Cosmetic Agents (third edition) published by the German Cosmetic Toiletry Perfumery and Detergent Association (IKW), Frankfurt.

Extracts of green tea, oak bark, stinging nettle, witch hazel, hops, henna, chamomile, burdock root, horsetail, whitethorn, lime-tree blossom, almond, aloe vera, pine needles, horsechestnut, sandalwood, juniper, coconut, mango, apricot, lime, wheat, kiwi, melon, orange, grapefruit, sage, rosemary, birch, hollyhock, cuckoo flower, wild thyme, yarrow, thyme, melissa, rest harrow, coltsfoot, marshmallow, meristem, ginseng and ginger root are preferred according to the invention. Extracts of green tea, oak bark, stinging nettle, witch hazel, hops, chamomile, burdock root, horsetail, lime-tree blossom, almond, aloe vera, coconut, mango, apricot, lime, wheat, kiwi, melon, orange, grapefruit, sage, rosemary, birch, cuckoo flower, wild thyme, yarrow, rest harrow, ginseng and ginger root are particularly preferred. Extracts of green tea, almond, aloe vera, coconut, mango, apricot, lime wheat, kiwi and melon and more particularly suitable. Water, alcohols and mixtures thereof may be used as extracting agents to prepare the aforementioned plant extracts. Of the group of alcohols, lower alcohols such as ethanol and isopropanol, but in particular polyhydric alcohols such as ethylene glycol and propylene glycol are preferred in this case, either as the sole extraction agent or mixed with water. Plant extracts based on water/propylene glycol in a ratio of from 1:10 to 10:1 have proved to be particularly suitable. The plant extracts may be used according to the invention in both pure and diluted form. If they are used in diluted form they conventionally contain approximately 2 to 80% by weight of the active substance and the extraction agent or extraction agent mixture used in the preparation thereof as a solvent. It may also be preferable to use mixtures of a plurality of, in particular two, different plant extracts.

To prevent the hair from becoming greasy again too quickly, some hair tonics contain substances such as specific tar ingredients, cysteinic acid derivatives or glycyrrhizin. However, the intended reduction in sebaceous gland production has not been demonstrated conclusively. In contrast, the effectiveness of anti-dandruff active ingredients has been proved beyond doubt. They are therefore used in appropriate hair tonics, among other hair care agents.

For the purposes of cleansing and nourishing facial skin in particular, there is a range of human skin care preparations available, such as facial toners, cleansing lotions, cleansing milks, cleansing creams and cleansing pastes. Some face packs cleanse the skin, but they generally refresh and care for facial skin. Facial toners are generally aqueous-alcohol solutions having a low surfactant content and further skin care substances. Cleansing lotions, milks, creams and pastes are generally based on O/W emulsions which have relatively low fatty component content and contain cleansing and nourishing additives. What are known as scruffing and scrub preparations contain substances which have a mild keratolytic effect to remove the upper layers of dead calloused skin, some of these preparations also additionally containing a powder with an abrasive effect. Agents for cleaning unclean skin also contain antibacterial and anti-inflammatory substances, since the accumulation of sebaceous material in comedones (blackheads) represents a breeding ground for bacterial infections and tends cause inflammation. The wide range of different skin cleansing products offered varies in its composition and content of different active ingredients depending on skin type and specific treatment purposes.

Bath additives for cleaning the skin in the bath or shower are widely used. Bath salts and tablets are intended to soften, color and fragrance the bath water and do not generally contain substances with a washing effect. By softening the bath water, these additives boost the cleaning power of soaps but their primary aim is to have a refreshing effect and to enhance the bath experience. Bath foams are of greater significance. If the additives have a higher content of lipid-replenishing and skincare substances, they are also known as cream baths.

The aforementioned cosmetic agents may be in different preparation forms. The most significant are hair and/or skin creams, skin lotions, oils and gels. Creams and lotions are based on emulsions in 0/W (oil in water) or W/O (water in oil) form. The main components of the oil or fat or lipid phase are fatty alcohols, fatty acids, fatty acid esters, waxes, vaselines, paraffins and further fat and oil components of predominantly natural origin. Besides water, the aqueous phase predominantly contains moisture-regulating and moisture-retaining substances as the main skincare agents and also contains consistency or viscosity-regulating agents. Further additives such as preservatives, antioxidants, complexing agents, perfume oils, dyes, or specific active ingredients are added to one of the two aforementioned phases, depending on the solubility and stability profiles thereof. The selection of the emulsifier system is crucial for the type of emulsion obtained and the properties thereof. Said emulsifier system can be selected in accordance with the HLB system.

The skincare agents may also contain further specific active ingredients such as milk protein products, egg yolk, lecithins, lipoids, phosphatides, cereal seed oils, vitamins—in particular vitamin F and biotin, which was previously called the skin vitamin (vitamin H)—and hormone-free placenta extracts.

Skin oils are one of the oldest types of skincare products and are still used today. They are based on non-drying plant oils such as almond oil or olive oil to which natural vitamin oils such as wheat germ oil or avocado oil and oily plant extracts from St. John's wort, chamomile, etc. are added. Skin gels are semi-solid transparent products which are stabilized by appropriate gel formers. This group is divided into three categories: oleogels (water-free), hydrogels (oil-free) and oil/water gels. The type of gel selected depends on the desired purpose. The oil/water gels have high emulsifier contents and have some advantages over emulsions from an aesthetic and use perspective.

The present invention further relates to washing and cleaning agents or cosmetic agents which contain iodide salts in combination with vanillin and/or vanillin derivatives. In this case, the washing and cleaning agents or cosmetic agents are preferably liquid or gel cleaners, softeners, washing agents, all-purpose cleaners, as well as skin creams, skin lotions, skin oils, gels, soaps and shampoos. The aforementioned agents preferably encompass at least one compound of formula I—

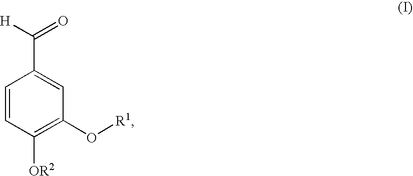

wherein R¹ is a methyl, ethyl or propyl residue and R² is hydrogen, a C₁-C₃ alkyl residue or —C(O)—R³, wherein R³ is an alkyl residue having 1 to 5 C atoms, preferably methyl, ethyl or n-propyl, isopropyl or butyl.

In a preferred embodiment R² is hydrogen or —C(O)—R³, wherein R³ is an isopropyl residue.

Preferred compounds in washing and cleaning agents or cosmetic agents according to formula I are selected from 4-hydroxy-3-methoxy-benzaldehyde (R¹=methyl, R²=H), 4-hydroxy-3-ethoxy-benzaldehyde (R¹=ethyl, R²=H), hydroxy-3-methoxy-benzaldehyde-2-methyl propionate (R¹=methyl, R²=—C(O)—CH(CH₃)₂).

As previously mentioned, the agents may also comprise further additives which may differ depending on requirements. The iodide salts in the washing and cleaning agents or cosmetic agents are preferably alkali metal iodides, which are preferably selected from calcium, potassium and/or sodium iodide.

In a further preferred embodiment, the washing and cleaning agents or cosmetic agents are soaps, shampoos or solid washing agent formulations (powders, granulates, tablets, tab-form) since the discoloration of vanillin or vanillin derivatives is particularly pronounced in these agents. As discussed above, bars of soap are particularly affected by this problem, so soaps containing iodide salts in combination with vanillin and/or vanillin derivatives are particularly preferred embodiments.

The present invention further relates to a method for inhibiting the discoloration of vanillin- and/or vanillin derivative-containing washing and cleaning agents or cosmetic agents, in particular soaps and/or solid washing agent formulations (powder, granulates, tablets, tab-form), in which alkali metal iodides, preferably selected from calcium, potassium and/or sodium iodide, are incorporated into the agent.

The invention will be described in greater detail by the following examples.

### EXAMPLES

The quantities used in the Examples are percentages by weight.

Bars of soap containing vanillin or vanillin derivatives according to formula I were produced by dissolving 3% of the respective (iodide) salt in 36% dipropylene glycol and optionally heating it slightly. This mixture was added to up to 61% of the respective perfume. The mixture was subsequently added to a perfume-free curd soap (talcum powder/soap 70/30) and kneaded. The finished bar of soap contained 1.5% of vanillin or vanillin derivatives. The bars of soap were stored under different conditions (temperature, UV, see Table) and the olfactory stability and change in color were subsequently determined. The results of the tests are shown in Table 1, wherein the following perfumes and scales were used—

Perfume 1: 4-hydroxy-3-ethoxy-benzaldehyde

Perfume 2: Hydroxy-3-methoxy-benzaldehyde-2-methyl propionate

Perfume 3: 4-hydroxy-3-methoxy-benzaldehyde

Olfactory stability (abbreviation: o.s.)—

1=very different

2=odorless

3=different

4=slightly different

5=O.K.

Discoloration—

0=no discoloration

1=slight discoloration

2=considerable discoloration

3=very strong discoloration

TABLE 1

| | Amount | 2 weeks at 23° C. | | 2 weeks at 40° C. | | 2 weeks - UV light | | 10 weeks at 23° C. | |
|---|---|---|---|---|---|---|---|---|---|
| | used | o.s. | Discoloration | o.s. | Discoloration | o.s. | Discoloration | o.S. | Discoloration |
| Perfume 1 | 1.50% | 5 | 1 | 4 | 1 | 4 | 2 | 5 | 2 |
| +Pot | 2.46% | 5 | 0 | 5 | 0 | 5 | 0-1 | 5 | 0 |
| +Cal | 2.46% | 5 | 0 | 5 | 0 | 5 | 0-1 | 5 | 0 |
| +Sod | 2.46% | 5 | 0 | 5 | 0 | 5 | 0-1 | 5 | 0 |
| Perfume 2 | 1.50% | 5 | 3 | 4 | 3 | 4 | 3 | 5 | 3 |
| +Pot | 2.46% | 5 | 0 | 5 | 0-1 | 5 | 1 | 5 | 0 |
| +Cal | 2.46% | 5 | 0 | 5 | 0-1 | 5 | 1 | 5 | 0 |
| +Sod | 2.46% | 5 | 0 | 5 | 0-1 | 5 | 1 | 5 | 0 |
| Perfume 3 | 1.50% | 5 | 3 | 4 | 3 | 4 | 3 | 5 | 3 |
| +Pot | 2.46% | 5 | 0-1 | 5 | 1 | 5 | 1 | 5 | 0-1 |
| +Cal | 2.46% | 5 | 0-1 | 5 | 1 | 5 | 1 | 5 | 0-1 |
| +Sod | 2.46% | 5 | 0-1 | 5 | 1 | 5 | 1 | 5 | 1 |

Table 1 shows that the soaps which contain iodide salts do not undergo discoloration or undergo only slight discoloration at different temperatures and under UV light. Even after 10 weeks at ambient temperature (23° C.) no soap discoloration was observed. In contrast, the soaps containing only vanillin or vanillin derivates and no iodide salts became discolored within 2 weeks. It was also found that these soaps did not exhibit any olfactory changes.

We claim:

1. Method of inhibiting discoloration in washing and cleaning agents and cosmetic agents comprising:
preparing a solution of an iodide salt,
adding the iodide salt solution to a fragrance mixture, and
incorporating the iodide salt solution and fragrance mixture into a washing and cleaning agent or cosmetic agent, thereby forming a washing and cleaning agent or cosmetic agent composition,
wherein the washing and cleaning agent or cosmetic agent composition containing the iodide salt undergoes little or no discoloration after two weeks at ambient temperature versus a washing and cleaning agent or cosmetic agent without the iodide salt.

2. Method of inhibiting discoloration according to claim 1 wherein the fragrance mixture comprises vanillin and/or vanillin derivatives.

3. Method of inhibiting discoloration according to claim 2 wherein the vanillin and/or vanillin derivatives are compounds according to the formula— 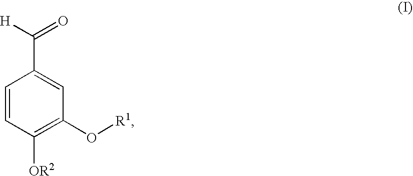
wherein R¹ is a methyl, ethyl or propyl residue and R² is hydrogen, a C₁-C₃ alkyl residue or —C(O)—R³, wherein R³ is an alkyl residue having 1 to 5 C atoms.

4. Method of inhibiting discoloration according to claim 2 wherein R² is hydrogen or C(O)—R³, and R³ is an isopropyl residue.

5. Method of inhibiting discoloration according to claim 2 wherein the vanillin and/or vanillin derivatives are chosen from 4-hydroxy-3-methoxy-benzaldehyde, 4-hydroxy-3-ethoxy-benzaldehyde and 4-hydroxy-3-methoxy-benzaldehyde-2-methyl propionate.

6. Method of inhibiting discoloration according to claim 1 wherein the fragrance mixture is a mixture of perfumes chosen from essential oils, perfume aldehydes, perfume ketones and/or perfume esters.

7. Method of inhibiting discoloration according to claim 7 wherein the mixture of perfumes is chosen from jasmones, ionones, damascones and damascenones, menthon, carvon, Iso E Super, methylheptenone, melonal, cymene, helional, hydroxycitronellal, Koavone, methyl nonyl acetaldehyde, phenylacetaldehyde, undecyl aldehyde, 3-dodecen-1-al, alpha-n-amylcinnamaldehyde, benzaldehyde, 3-(4-tert-butylphenyl)-propanal, 2-methyl-3-(paramethoxyphenyl propanal), 2-methyl-4-(2,6,6-trimethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzaldehyde, 2,4-dimethyl-3-cyclohexene-1-carboxaldehyde, 2-methyl-3-(isopropylphenyl) propanal, decylaldehyde, 2,6-dimethyl-5-heptenal, alpha-n-hexylcinnamaldehyde, 7-hydroxy-3,7-dimethyloctanal, undecenal, 2,4,6-trimethyl-3-cyclohexene-1-carboxaldehyde, 1-dodecanal, 2,4-dimethyl-cyclohexene-3-carboxaldehyde, 4-(4-hydroxy-4-methylpentyl)-3-cyclohexene-1-carboxaldehyde, 2-methylundecanal, 2-methyldecanal, 1-nonanal, 1-octanal, 2,6,10-trimethyl-5,9-undecadienal, 2-methyl-3-(4-tert-butyl)propanal, dihydrocinnamaldehyde, 3,7-dimethyloctan-1-al, 1-undecanal, 10-undecen-1-al, 4-hydroxy-3-methoxybenzaldehyde, trans-4-decenal, 2,6-nonadienal, para-tolyl acetaldehyde, 3,7-dimethyl-2-methylene-6-octenal, 2-methyloctanal, alpha-methyl-4-(1-methylethyl)benzeneacetaldehyde, 2-methyl-3-phenyl-2-propen-1-al, 3,5,5-trimethylhexanal, 3-propyl-bicyclo[2.2.1]-hept-5-ene-2-carbaldehyde, 9-decenal, 3-methyl-5-phenyl-1-pentanal, methyl nonyl acetaldehyde, citral, 1-decanal, Florhydral, 2,4-dimethyl-3-cyclohexene-1-carboxaldehyde and heliotropin.

8. Method of inhibiting discoloration according to claim 1 wherein the agent is a washing and cleaning agent and the washing and cleaning agent is a liquid or gel cleaner, softener, washing agent or all-purpose cleaner.

9. Method of inhibiting discoloration according to claim 1 wherein the agent is a cosmetic agent and the cosmetic agents is a skin cream, skin lotion, skin oil, gel, soap or shampoo.

10. Method of inhibiting discoloration according to claim 1 wherein the iodide salts are alkali metal iodide salts.

11. Method of inhibiting discoloration according to claim 9 wherein the alkali metal iodide salts are chosen from calcium, potassium and/or sodium iodide.

12. Method of inhibiting discoloration according to claim 1 wherein the iodide salt is present in the washing and cleaning agent or cosmetic agent in an amount of from 0.05 to 5% by weight, based on total weight of the composition.

13. Washing and cleaning or cosmetic composition comprising iodide salts and vanillin and/or vanillin derivatives.

14. Washing and cleaning or cosmetic composition according to claim 12, wherein the composition is a washing and cleaning composition chosen from liquid or gel cleaners, softeners, washing agents, and all-purpose cleaners.

15. Washing and cleaning or cosmetic composition according to claim 12, wherein the composition is a cosmetic composition chosen from skin creams, skin lotions, skin oils, gels, soaps or shampoos.

16. Washing and cleaning or cosmetic composition according to claim 12, wherein the iodide salts are alkali metal iodides.

17. Washing and cleaning or cosmetic composition according to claim 15, wherein the alkali metal iodides are chosen from calcium, potassium and/or sodium iodide.

18. Washing and cleaning or cosmetic composition according to claim 12, wherein the iodide salts are present in an amount of from 0.05 to 5% by weight, based on total weight of the composition.

19. Washing and cleaning or cosmetic composition according to claim 12, wherein the composition is a cosmetic soap and further comprises alkali salts of fatty acids.
